(19) United States
(12) Patent Application Publication
(10) Pub. No.: US 2002/0016296 A1
(43) Pub. Date: Feb. 7, 2002

(54) Aspartylprotease

(76) Inventors: Christian Haass, Icking (DE); Harald Steiner, Muenchen (DE); Katja Fechteler, Wiesbaden (DE); Marcus Kostka, Mainz (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 09/896,053

(22) Filed: Jun. 29, 2001

Related U.S. Application Data
(60) Provisional application No. 60/224,161, filed on Aug. 9, 2000.

(30) Foreign Application Priority Data
Jul. 7, 2000 (DE) DE 100 32 709.5

(51) Int. Cl. A61K 38/08; C07K 7/06

(52) U.S. Cl. 514/16; 530/329

(57) ABSTRACT

The invention relates to proteins, peptides or aspartyl-proteases comprising specified consensus motifs and nucleic acids encoding said proteins, peptides or aspartyl-proteases. The invention further relates to methods of screening for substances capable of inhibiting said aspartyl-proteases, substances identifiable with said method and pharmaceutical compositions comprising said substances.

### RELATED APPLICATION

The benefit of prior provisional application Ser. No. 60/224,161, filed Aug. 9, 2000 is hereby claimed.

### BACKGROUND

The invention relates to proteins or, peptides (e.g. aspartyl-proteases) comprising specified consensus motifs and nucleic acids encoding said proteins, peptides or aspartyl-proteases. The invention further relates to methods of screening for substances capable of inhibiting said aspartyl-proteases, substances identifiable with said method and pharmaceutical compositions comprising said substances. In particular, the invention belongs to the field of neurodegenerative diseases and Alzheimer's disease.

Presenilin (PS) proteins play a central role in Alzheimer's disease (AD). Familial AD (FAD) associated mutations affect the endoproteolytic generation of Amyloid β-peptide (Aβ). By an unknown mechanism these mutations shift the γ-secretase cleavage by two amino acids, which results in the enhanced production of the highly amyloidogenic 42 amino acid residue Aβ (Aβ42) (Selkoe, D. J., 1999; Nature 399:A23-31). Since the longer version of Aβ is known to precipitate much faster as Aβ40 (Lansbury, Jr., P. T., 1997; Neuron 19:1151-4), it is widely believed that early onset AD is caused by the increased deposition of amyloid plaques. A similar pathogenic mechanism of early amyloid plaque deposition was described for almost all FAD related Amyloid β precursor protein (βAPP) mutations (Selkoe, D. J., 1999; Nature 399:A23-31).

PS proteins undergo endoproteolysis (Thinakaran, G., et al, 1996; Neuron 17:181-90) and exist as a complex composed of N- and C-terminal fragments (NTF and CTF; (Capell, A. et al, 1998; J Biol Chem 273:3205-11; Yu, G. et al., 1998; J Biol Chem 273:16470-5; Thinakaran, G. et al., 1998; Neurobiol Dis 4:438-53). Complex formation appears to be a prerequisite for PS function (Steiner, H. et al., 1998; J Biol Chem 273:32322-31; Saura, C. A. et al., 1999; J Biol Chem 274:13818-23). PS proteins are required to support the cleavage of membrane bound proteins either within or close to the cytoplasmic site of the membrane (Brown, M. S. et al, 2000; Cell 100:391-8; Haass, C., DeStrooper, B., 1999; Science 286:916-9). Several substrates have been identified, which include members of the APP family, Notch, and Ire1, and a PS1 gene ablation results in the inhibition of endoproteolysis of these proteins (Niwa, M. et al., 1999; Cell 99:691-702; DeStrooper, B. et al., 1998; Nature 391:387-390; DeStrooper, B. et al., 1999; Nature 398:518-22). Moreover, in the PS1 knock out a Notch-like phenotype is observed (Shen, J. et al., 1997; Cell 89:629-39; Wong, P. C. et al., 1997; Nature 387-288-92), which is strongly augmented if in addition to PS1 the homologous PS2 is deleted as well (Donoviel, D. B. et al., 1999; Genes Dev 13:2801-1; Herreman, A. et al., 1999; Proc Natl Acad Sci USA 96:11872-7). Such a double knock out of both presenilins results in a complete inhibition of Aβ generation and Notch endoproteolysis (Zhang, Z. et al., 2000; Nature Cell Biol 2:463-465; Herreman, A. et al., 2000; Nature Cell Biol 2:461-2). Furthermore, human presenilins rescue the Notch phenotype of sel-12 mutant worms (Levitan, D. et al., 1996; Proc Natl Acad Sci USA 93:14940-4; Baumeister, R. et al., 1997; Genes Funct 1:149-59; Steiner, H. et al., 1999; J Biol Chem 274:28669-73) further supporting the finding that presenilins are involved in Notch signaling.

Recent evidence suggests that critical aspartate residues of PS1 (Wolfe, M. S. et al., 1999; Nature 398:513-7) and PS2 (Steiner, H. et al., 1999; J Biol Chem 274:28669-73, Kimberly, W. T. et al., 2000; J Biol Chem 275:3173-8) are functionally required for proteolysis of PS targets and these aspartates are conserved during evolution (Leimer, U. et al., 1999; Biochemistry 38:13602-9). Wolfe et al. (Wolfe, M. S. et al., 1999; Nature 398:513-7) demonstrated that the mutagenesis of these aspartate residues in PS1 results in the accumulation of βAPP CTFs, which are believed to be the precursors for the ultimate γ-secretase cleavage. Moreover, a significant reduction of Aβ generation was observed, which appeared to be correlated with the lack of endoproteolysis of the aspartate mutant PS derivative (Wolfe, M. S. et al., 1999; Nature 398:513-7). Based on their findings, Wolfe et al. (Wolfe, M. S. et al., 1999; Nature 398:513-7) postulated that PS1 may be an aspartyl protease which is identical to the γ-secretase. This hypothesis is supported by the recent finding that γ-secretase inhibitors can be cross-linked to the proteolytic fragments of PS1 and PS2 (Li, Y. -M., et al., 2000; Nature 405:689-94, Esler, W. P., et al., 2000; Nature Cell Biol 2:428-433). However, no homology to any known protease was so far detected ( Wolfe, M. S. et al., 1999; Nature 398:513-7). Moreover, it is of particular concern that no consensus protease active site motif was observed around the predicted active sites of presenilins. Therefore, only indirect evidence exists to support the hypothesis that PS proteins are proteolytic enzymes or are even identical with the γ-secretase itself. Thus, the problem underlying the present invention was the need in the art for the protease per se and the protease motifs thereof.

### SUMMARY OF THE INVENTION

The above-captioned technical problem is solved by the embodiments characterized in the claims and the description.

The invention relates to proteins, peptides or aspartyl-proteases comprising specified consensus motifs and nucleic acids encoding said proteins, peptides or aspartyl-proteases. The invention further relates to methods of screening for substances capable of inhibiting said aspartyl-proteases, substances identifiable with said method and pharmaceutical compositions comprising said substances.

### DESCRIPTION OF THE FIGURES

FIG. 1: (A) Schematic representation of PS1.The critical aspartates in transmembrane (TM) 6 and 7 (arrow heads) as well as the endoproteolytic cleavage site(s) (black box) and the mutations introduced at amino acid residue 384 are indicated. (B) Expression, endoproteolytic processing and replacement of the mutant PS1 derivatives. Upper panel: Cell lysates were immunoprecipitated with antibody 3027 and precipitated PS1 derivatives (CTFs and holoprotein) were visualized by immunoblotting using antibody BI.3D7 (Steiner, H. et al., 1999; J. Biol Chem 274:7615-8). Note that endoproteolysis of PS1 is diminished in cell lines expressing the G384K, G384D, and D385A mutant. Lower panel: Overexpressed mutant PS1 derivatives displace endogenous PS2 CTFs. Lysates from the cell lines described in (B) were immunoprecipitated with antibody 3711 and immunoblotted using the monoclonal antibody BI.HF5c (Steiner, H., et al., 1999; J Biol Chem 274:7615-8). (C) Accumulation of βAPP-CTFs in G384 mutant cell lines. Aliquots of lysates used in (B) were immunoblotted with antibody 6687 to the C-terminus of βAPP (Steiner, H., et al., 1999; J Biol Chem 274:7615-8). βAPP-CTFs generated by α-, and β-secretase (longer exposure reveals significant amounts of β-secretase generated βAPP CTFs) strongly accumulated in cells expressing the PS1 G384 artificial mutants as well as in cells expressing PS1 D385A as observed previously (Wolfe, M. S. et al., 1999; Nature 398:513-7, Steinter, H., et al., 1999; J Biol Chem 274:7615-8).

FIG. 2: Effects of the mutant PS 1 derivatives on APP endoproteolysis. (A) Upper panel (conditioned media): Aβ species were immunoprecipitated from conditioned media of metabolically labeled cells with antibody 3926 and separated on a previously described Tris-Bicine gel system, which allows the specific identification of Aβ40 and Aβ42 (Wiltfang, J. et al., 1997; Electrophoresis 18:527-32). APPs were immunoprecipitated with antibody 5313 (Steiner, H., et al., 1999; J Biol Chem 274:7615-8). Lower panel (cell lysates): Full length APP (N′/O′-glycosylated APP and N′-glycosylated APP) was immunoprecipitated from cell lysates with antibody 5313 (Steiner, H., et al., 1999; J Biol Chem 274:7615-8). (B) Quantitation of Aβ levels by phosphorimaging. Aβ species were immunoprecipitated as in (A) but separated on 10-20% Tris-Tricine gels. Total Aβ levels are expressed relative to the Aβ levels in PS1 wild type (wt) expressing cells. Bars represent the mean +/−S.E. of three independent experiments. (C) Independent quantitation of total Aβ levels by a previously described ELISA (Steiner, H. et al (1998) J. Biol Chem 273:32322-31). Horizontal bars represent the mean of six independent experiments using conditioned media from unlabeled cells. (D) Quantitation of the Aβ42/Aβtotal ratio (Aβtotal=Aβ40+Aβ42) by phosphorimaging. AP species were identified as described in (A). Bars represent the mean +/−S.E. of three independent experiments. Insert: Aβ42/Aβ total ratio (Aβtotal=Aβ40+Aβ42) determined in conditioned media from cells expressing PS1 wt, PS1 Δexon 9 (Steinter, H., et al., 1999; J Biol Chem 274:7615-8).

FIG. 3: Effects of the mutant PS1 derivatives on Notch endoproteolysis. Cell lines expressing PS1 derivatives were transfected with the NotchΔE cDNA (Schroeter, E. H. et al., 1998; Nature 393:382-6). Notch C-terminal domain (NICD) formation was analyzed in pulse chase experiments as described (Steiner, H. et al., 1999; J Biol Chem 274:28669-73). Note the significant change of the ratio of NΔE:NICD in cells expressing the G384P and G384K mutation as compared to those lines, which express wt PS 1 or PS1 G384A. Consistent with previous results, NICD formation was blocked in cell lines expressing PS 1 D385A (Steiner, H. et al., 1999; J Biol Chem 274:28669-73, Capell, A. et al., 2000; Nat Cell Biol 2:205-11). As observed previously (Baumeister, R. et al., 1997; Genes Funct 1:149-59, Song, W. et al., 1999; Proc Natl Acad Sci USA 96:6959-63, Schroeter, E. H. et al., 1998; Nature 393:382-6), NΔE is not only processed to NICD but also degraded during the cold chase.

FIG. 4: Sequence comparison of the active site consensus sequence of PSs (A) with members of the TFPP family (B). Hs=homo sapiens; Bt=Bos taurus; Rn=Rattus norwegicus; Mm=Mus musculus; X1=Xenopus laevis; Dr=Danio rerio; Dm=Drosophila melanogaster; At=Arabidopsis thaliana; Ce=Caenorhabditis elegans; for the abbreviations of the bacterial type 4 prepilin peptidases (TFPPs) see (LaPointe, C. F. and Taylor R. K., 2000; J Biol Chem 275:1502-10). The conserved glycine as well as the aspartate residue are highlighted. For details see text. Arrowheads indicate the critical aspartate residues of PSs and TFPPs.

### DETAILED DESCRIPTION OF THE INVENTION

Before the embodiments of the present invention it must be noted that as used herein and in the appended claims, the singular forms “a”, “an”, and “the” include plural reference unless the context clearly dictates otherwise. Thus, for example, reference to “an aspartyl-protease” includes a plurality of such aspartyl-proteases, reference to the “cell” is a reference to one or more cells and equivalents thereof known to those skilled in the art, and so forth. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention. The amino acid abbreviations are according to the standard three letter code.

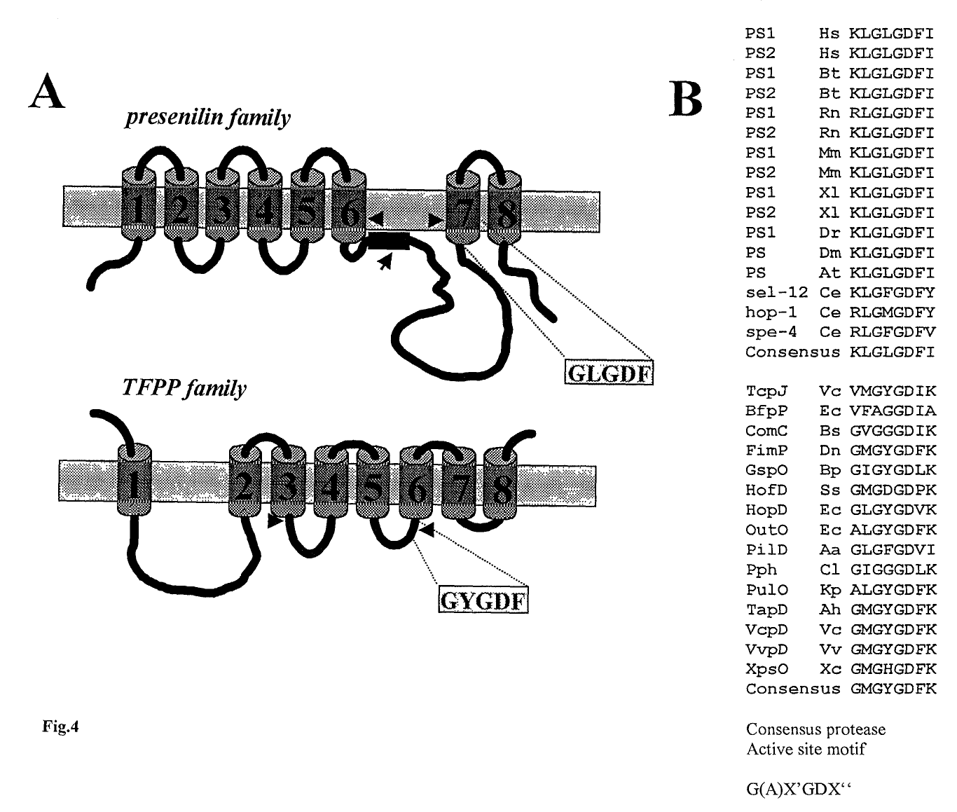

The invention provides a protein or peptide comprising the consensus motif X¹-Leu-Gly-X²-Gly-Asp-Phe-X³ or X⁴-X⁵-X⁶-X⁷-Gly-Asp-X⁸-X⁹, wherein X¹, is Lys or Arg; X² is Lys, Phe, Met or Leu; X³ is Ile, Tyr or Val; X⁴ is Val, Gly, Ala; X⁵ is Met, Phe, Val, Ile, Leu; X⁶ is Gly or Ala, X⁷ is Tyr, Gly, Asp, Phe or His; X⁸ is Ile, Leu, Pro, Val or Phe; and X⁹ is Lys, Ala or Ile. In addition to the consensus motif, the protein or peptide comprises further amino acids and may also be glycosylated. Most preferably, the protein or peptide is an aspartyl-protease. These consensus motifs are exemplified in FIG. 4 and comprise the active site of e.g. proteases of the γ-secretase, presenilin or type 4 prepilin peptidase (TFPP) families.

In a more preferred embodiment, the invention relates to a protein or peptide according to the invention comprising the consensus motif characterized by the amino acids X¹-Leu-Gly-X²-Gly-Asp-Phe-X³ wherein X¹, is Lys or a Arg; X² is Lys, Phe, Met or Leu and X³ is Ile, Tyr or Val. The consensus motif is exemplified in FIG. 4 (upper part) and comprises the active site of e.g. proteases of the γ-secretase or presenilin family.

In another more preferred embodiment, the invention relates to a protein or peptide according to the invention comprising the consensus motif characterized by the amino acids X⁴-X⁵-X⁶-X⁷-Gly-Asp-X⁸-X⁹, wherein X⁴ is Val, Gly, Ala; X⁵ is Met, Phe, Val, Ile, Leu; X⁶ is Gly or Ala, X⁷ is Tyr, Gly, Asp, Phe or His; X⁸ is Ile, Leu, Pro, Val or Phe and X⁹ is Lys, Ala or Ile. The consensus motif is exemplified in FIG. 4 (lower part) and comprises the active site of e.g. proteases of the TFPP family.

In another more preferred embodiment, the invention relates to a peptide according to the invention consisting of the the consensus motif characterized by the amino acids X¹-Leu-Gly-X²-Gly-Asp-Phe-X³, wherein X¹, is Lys or a Arg; X² is Lys, Phe, Met or Leu and X³ is Ile, Tyr or Val. The peptide consisting of the consensus motif of e.g. proteases of the γ-secretase or presenilin family is depicted in FIG. 4 (upper part).

In another more preferred embodiment, the invention relates to a peptide according to the invention consisting of the the consensus motif characterized by the amino acids X⁴-X⁵-X⁶-X⁷-Gly-Asp-X⁸-X⁹, wherein X⁴ is a Val, Gly, Ala; X⁵ is a Met, Phe, Val, Ile, Leu; X⁶ is a Gly or Ala, X⁷ is a Tyr, Gly, Asp, Phe or His; X⁸ is a Ile, Leu, Pro, Val or Phe and X⁹ is Lys, Ala or Ile. The peptide consisting of the consensus motif of e.g. proteases of the TFPP family is depicted in FIG. 4 (lower part).

In another more preferred embodiment, the invention relates to a peptide according to the invention consisting of the the consensus motif Lys-Leu-Gly-Leu-Gly-Asp-Phe-Ile. The peptide consisting of the consensus motif of e.g. proteases of the γ-secretase or presenilin family is depicted in FIG. 4 (upper part).

In another more preferred embodiment, the invention relates to a peptide according to the invention consisting of the the consensus motif Gly-Met-Gly-Tyr-Gly-Asp-Phe-Lys. The peptide consisting of the consensus motif of e.g. proteases of the TFPP family is depicted in FIG. 4 (lower part).

In another more preferred embodiment, the invention relates to a protein according to the invention characterized in that it is an aspartyl-protease.

In still another more preferred embodiment, the invention relates to an aspartyl-protease according to the invention comprising the consensus motif Lys-Leu-Gly-Leu-Gly-Asp- Phe-Ile. The consensus motif is exemplified in FIG. 4 (upper part) and comprises the active site of e.g. aspartyl-proteases of the γ-secretase or presenilin family.

In still another more preferred embodiment, the invention relates to an aspartyl-protease according to the invention comprising the consensus motif Gly-Met-Gly-Tyr-Gly-Asp-Phe-Lys. The consensus motif is exemplified in FIG. 4 (lower part) and comprises the active site of e.g. aspartyl-proteases of the TFPP family.

In still another more preferred embodiment, the invention relates to a aspartyl-protease comprising the consensus motif characterized by the amino acids Gly-X¹-X²-Gly-Asp-X³; wherein X¹ is Ala or no amino acid (Ala is an optional amino acid, i.e. either Ala is at this position or the sequence continues with X²); X² is any amino acid and X³ is Phe, Ile, Val or Leu. This aspartyl-protease comprises e.g. the consensus motif for any aspartyl-protease comprising the γ-secretase, presenilin and TFPP families (see example 1).

In still another more preferred embodiment, the invention relates to a peptide consisting of the consensus motif characterized by the amino acids Gly-X¹-X²-Gly-Asp-X³; wherein X¹ is Ala or no amino acid; X² is any amino acid and X³ is Phe, Ile, Val or Leu. This peptide consists of e.g. the consensus motif for any aspartyl-protease comprising the γ-secretase, presenilin and TFPP families (see example 1).

A further aspect of the present invention is a nucleic acid encoding any one of the proteins or peptides according to the invention. This means a DNA or RNA molecule encoding any of the proteins, peptides or aspartyl-proteases as described above, in the Figures or in the examples.

Yet another important embodiment of the present invention is a method of screening for substances capable of inhibiting an aspartyl-protease according to the invention as disclosed above, comprising:

(a) culturing cells that express (i) an aspartyl-protease according to the invention, and

(ii) a membrane-associated fusion protein comprising a substrate with the specific cleavage site of said aspartyl-protease and a reporter;

(b) incubating said cells with a test substance;

(c) measuring the amount of cleaved-off reporter, and

(d) comparing the value obtained in step (c) to the value obtained in the absence of the test compound.

Membrane-associated according to the invention means that the substrate is bound to the surface of the membrane or to integrated membrane proteins. These substrates also include substrates which interact with the hydrophobic part of the membrane via chemical groups which were added by post-translational modifications. Membrane-associated substrates further include substrates which interact with the hydrophobic part of the membrane via amino acid side chains, even though less than integrated membrane proteins. Substrate relates to peptides and proteins which at least contain one cleavage site of said protease. Said substrates may also be modified, e.g. glycosylated (Sambrook and Maniatis (1989). Molecular Cloning:A Laboratory Manual, Cold Spring Harbor Laboratory, Cold Spring Harbor, N.Y.). According to the invention, the substrate is fused to a reporter which is used to detect the activity of the aspartyl-protease (cleavage). The fusion proteins according to the invention can be generated by standard molecular biological methods (Sambrook and Maniatis (1989, see above). The DNA encoding a reporter protein may be commercially available, e.g. Clontech, Heidelberg and may be inserted by standard methods into suitable vectors for expression. The DNA encoding the substrate may be derived by standard methods and is available from suitable gene banks. Test substances may be any substance, e.g. a protein or chemical compound known to the artisan or new substances. Said test substances are available e.g. from commercial substance libraries. By comparing the value obtained in the absence of the test substance with the value obtained in the presence of the test substance according to step (d) the specifity of the method can be controlled. By using such a control, the person skilled in the art can recognize the substances which inhibit the aspartyl-protease according to the invention.

A suitable cell or cell line, preferably a eukaryotic cell or a cell line, to be transformed with nucleic acid constructs to express the aspartyl-protease according to the invention may be any cell or cell line known to the expert in the field, in particular cells or cell lines used in neurological and neurobiology research. Examples of such cells or cell lines useful for producing the transformed cell lines of the invention include mammalian cells or cell lines (e.g. the cell lines human embryonic kidney (HEK) 293, BHK, GH3, H4, U373, NT2, PC12, COS, CHO, Ltk, fibroblasts, myelomas, neuroblastomas, hybridomas, öocytes, embryonic stem cells), insect cell lines (e.g., using baculovirus vectors such as pPbac or pMbac (Stratagene, La Jolla, USA)), yeast (e.g., Pichia pastoris or using yeast expression vectors such as pYESHIS (Invitrogen, San Diego, USA)), and fungi.

Examples of suitable reporter genes include, but are not limited to E. coli β-galactosidase (β-gal, Luban and Goff, 1995; Curr Opin Biotechnol 6, 59-64), xanthine-guanine phosphoribosyl transferase (Chu and Berg, 1985; Nucleic Acids Res 13:2921-2930), galactokinase (Schumperli et al., 1982; Proc Natl Acad Sci USA 79:257-261), interleukin-2 (Cullen, 1986; Cell 46:973-982), thymidine kinase (Searle et al., 1985; Mol Cell Biol 5:1480-1489), alkaline phosphatase (Toh et al., 1989; Eur J Biochem 182:231-237; Henthorn et al., 1988; Proc Natl Acad Sci USA 85:6342-6346), secretory alkaline phosphatase (SEAP) or secreted placental alkaline phosphatase (Berger et al., 1988, Gene 66:1-10) and chloramphenicol-acetyltransferase (CAT, Alton and Vapnek, 1979, Nature 282:864-869; Gorman et al., 1982, Mol Cell Biol 2:1044-1051; Tsang et al., 1988, Proc Natl Acad Sci USA 85:8598-8602) green fluorescent protein (GFP) produced by the bioluminescent jellyfish (Chalfie et al., 1994, Science 263:802-805), derivatives thereof such as blue fluorescent protein (BFP), firefly luciferase (deWet et al., 1987, Mol Cell Biol 7:725-737; Engebrecht and Silverman, 1984, Proc Natl Acad Sci USA 81:4154-4158). Many of these and other useful reporter genes are available from commercial sources. Expression products of the reporter genes such as reporter enzymes can be measured using standard methods. For example, bioassays can be carried out for biologically active proteins such as interleukin-2. Enzyme assays can be performed when the reporter gene product is a reporter enzyme such as alkaline phosphatase or β-galactosidase. Alternatively, various types of immunoassays such as competitive immunoassays, direct immunoassays and indirect immunoassays may be used. Such immunoassays involve the formation of immune complexes containing the reporter gene product and a measurable “reporter” or a “label”. As used herein, the term “reporter” includes moieties that can be detected directly, such as fluorochromes and radiolabels, and moieties such as enzymes that must be reacted or derivatized to be detected.

Yet another important embodiment of the present invention is a method according to the invention, comprising culturing cells that express a fusion protein comprising the cleavage site of the γ-secretase. The fusion protein according to the invention is membrane-associated or membrane-integrated.

Yet another important embodiment of the present invention is a method according to the invention, comprising culturing cells that express a fusion protein comprising the cleavage site of the presenilinase.

Yet another important embodiment of the present invention is a method according to the invention, characterized in that the fusion protein comprises amyloid β, This is the substrate of the γ-secretase.

Yet another important embodiment of the present invention is a method according to the invention, characterized in that the fusion protein comprises a fragment of amyloid β. This fragment comprises the substrate of the γ-secretase and must at least contain one specific cleavage site of the γ-secretase.

Yet another important embodiment of the present invention is a method according to the invention, characterized in that the fusion protein comprises the amyloid precursor protein or a fragment thereof.

Presenilin cleavage can occur either autoproteolytically or by the presenilinase. Yet another important embodiment of the present invention is a method according to the invention, characterized in that the fusion protein comprises presenilin 1 or a fragment thereof. The fragments may be N-terminal fragments (NTF) of approx. 21-28 kDa or C-terminal fragments (CTF) of 16-24 kDa (Haas et al., 1998; J Neural Transm Suppl 53:159-67; Okochi et al., 1997; FEBS Lett 418:162-6; Thinakaran et al., 1996 (Ref. 3) which have to go beyond the presenilinase cleavage site. Presenilin 1 or fragments thereof are the substrates of the presenilinase and must at least contain one specific presenilinase cleavage site.

Yet another important embodiment of the present invention is a method according to the invention, characterized in that the fusion protein comprises presenilin 2 or a fragment thereof. The fragments may be N-terminal fragments (NTF) of approx. 28-30 kDa or C-terminal fragments (CTF) of 20-25 kDa (Haas et al., 1998; J Neural Transm Suppl 53:159-67; Kim et al., J Biol Chem 1997, 272, 11006-11010; Podlisny et al., 1997; Neurobiol Dis 3:325-337) which have to go beyond the presenilinase cleavage site. Presenilin 2 or fragments thereof are the substrates of the presenilinase and must at least contain one specific presenilinase cleavage site.

Yet another important embodiment of the present invention is a method according to the invention, characterized in that the method is a high throughput screening (HTS) method. HTS relates to an experimental setup wherein a large number of compounds are tested simultaneously. Preferably, said HTS setup may be carried out in microplates, may be partially or fully automated and may be linked to electronic devices such as computers for data storage, analysis, and interpretation using bioinformatics. Preferably, said automation may involve robots capable of handling large numbers of microplates and capable of carrying out several thousand tests per day. Preferably, a test compound which shows a desired inhibitory function in a cell-free system will also be tested in a cell-based system using a cell line according to the present invention. The term HTS also comprises ultra high throughput screening formats (UHTS). Preferably, said UHTS formats may be carried out using 384- or 1536-well microplates, sub-microliter or sub-nanoliter pipettors, improved plate readers and procedures to deal with evaporation. HTS methods are described for example in U.S. Pat. No. 5,876,946 A or U.S. Pat. No. 5,902,732 A. The expert in the field can adapt the method described below to a HTS or UHTS format without the need of carrying out an inventive step.

Another preferred embodiment of the present invention is the use of a protein or peptide according to the invention for the identification of an inhibitor of the presenilinase or the autoproteolytic cleavage of presenilin. Such a method may comprise:

(a) culturing cells expressing (i) the protein or pepetide according to claim 1 and (ii) a membrane-associated fusion protein comprising a substrate with the specific cleavage site of said presenilinase and a reporter;

(b) incubating said cells with a test substance;

(c) measuring the amount of cleaved-off reporter; and

(d) comparing the value obtained to the value obtained in the absence of the test compound.

Another preferred embodiment of the present invention is the use of a protein or peptide according to the invention for the identification of an inhibitor of the γ-secretase. Such a method may comprise:

(a) culturing cells expressing (i) the protein or pepetide according to claim 1 and (ii) a membrane-associated fusion protein comprising a substrate with the specific cleavage site of said γ-secretase and a reporter;

(b) incubating said cells with a test substance;

(c) measuring the amount of cleaved-off reporter; and

(d) comparing the value obtained to the value obtained in the absence of the test compound.

Yet another preferred embodiment of the present invention is a substance identifiable with a method according to the invention (see above), characterized in that it is capable of specifically inhibiting the proteolytic cleavage of a γ-secretase-substrate.

Yet another preferred embodiment of the present invention is a substance identifiable with a method according to the invention (see above), characterized in that it is capable of specifically inhibiting the proteolytic cleavage of a presenilinase-substrate. In a most preferred embodiment, said substrate is presenilin.

Said substance may be a protein or a chemical compound. An example for such a substance includes, but is not limited to the substance shown below:

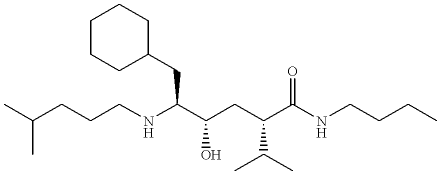

Another important aspect of the present invention is the use of a substance according to the invention (see above) for the manufacture of a medicament in the treatment of neurodegenerative diseases, preferably Alzheimer's disease. Use of such a substance may include administering said substance to an individual for treating a neurodegenerative disease, including but not limited to AD. One skilled in the art would be able to determine route and dosage based on disease, condition of individual, etc.

Another preferred embodiment of the present invention is a pharmaceutical composition characterized in that it comprises a substance according to the invention, and optionally pharmaceutically acceptable carriers or excipients. A pharmaceutically acceptable carrier may contain physiologically acceptable compounds that act, for example, to stabilize or to increase the absorption of an substance capable of inhibiting the proteolytic cleavage of a presenilinase or γ-secretase-substrate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients (see also e.g. Remington's Pharmaceutical Sciences (1990), 18th ed. Mack Publ., Easton). One skilled in the art would know that the choice of a pharmaceutically acceptable carrier, including a physiologically acceptable compound, depends, for example, on the route of administration of the composition.

### EXAMPLE 1

Immediately adjacent to the critical aspartate at residue 385 of human PS1 an FAD associated mutation (PS1 G384A) has been identified (Cruts, M. et al., 1995; Hum Mol Genet 4:2363-71, Tanahashi, H. et al., 1996; Neurosci Lett 218:139-41). Since that mutation occurs so close to the critical aspartate residue 385 and results in an increase of Aβ42 generation (DeJonghe, C. et al., 1999; Neurobiol Dis 6:280-7, Murayama, O. et al., 1999; Neurosci Lett 265:61-3), we hypothesized that the glycine may have an important function in addition to the critical aspartate at position 385 and may affect the putative active site of presenilins. We, therefore, mutagenized residue 384 by inserting a variety of different amino acids and analyzed PS endoproteolysis and γ-secretase function.

Mutations at Amino Acid 384 Affect PS1 Endoproteolysis and βAPP CTF Generation

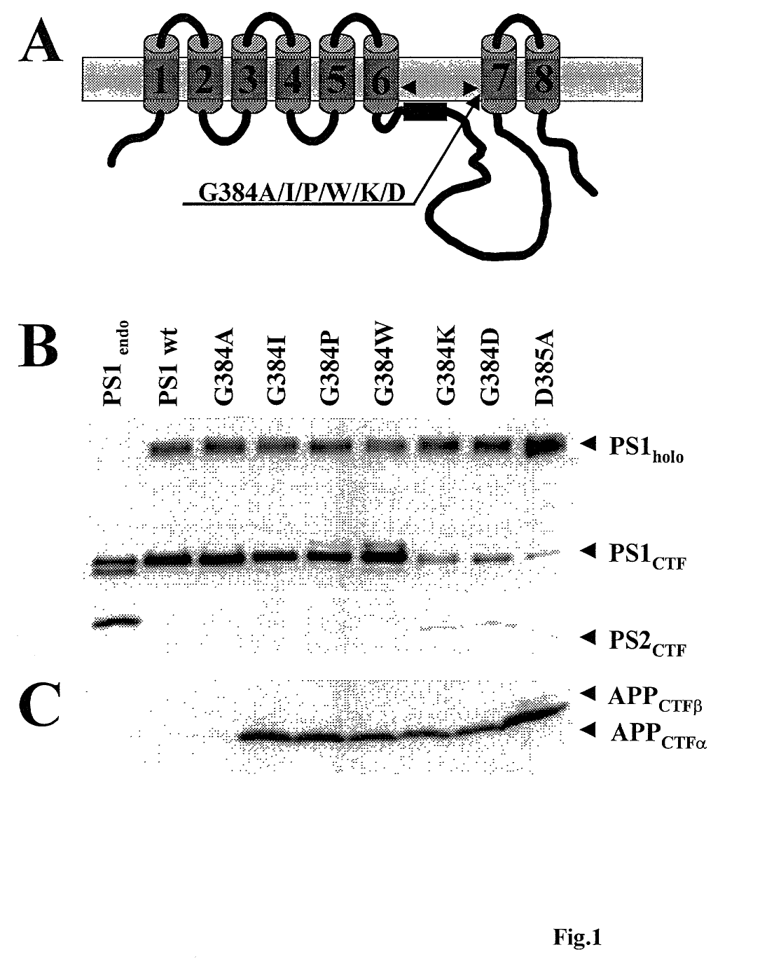

The glycine at residue 384 was exchanged to amino acids containing aliphatic-, α-helix breaking-, aromatic-, as well as charged (positive and negative) side chains (cDNAs encoding PS1 G384A, PS1 G2861, PS1 G286P, PS1 G384W, PS1 G384K and PS1 G384D were constructed by oligonucleotide-directed mutagenesis using the polymerase chain reaction as described previously (Steiner, H. et al., 1999; Biochemistry 38:14600-5). The respective PCR products were cloned into EcoRI/Xhol restriction sites of the expression vector pcDNA3.1 containing a zeocin resistance gene (Invitrogen) and sequenced to verify successful mutagenesis.) (FIG. 1A). These PS1 derivatives were stably transfected into K293 cells overexpressing Swedish mutant βAPP (HEK 293 cells stably expressing PS 1 G384A, PS1 G2861, PS1 G286P, PS1 G38W, PS1 G384K, PS1 G384D were generated by transfection of HEK 293 cells stably expressing βAPP containing the Swedish mutation (Citron, M. et al., 1992; Nature 360:672-4). Transfected HEK 293 cells were cultured in DMEM supplemented with 10% fetal bovine serum, 1% penicillin/streptomycine, 200μg ml⁻¹ G418 (to select for βAPP expression) and 200 μg ml⁻¹ zeocin (to select for presenilin expression)). This cell line was used in numerous previous reports to analyze βAPP processing [(Steiner, H. et al., 1999; Biochemistry 38:14600-5) and citations therein] as well the function of presenilins in βAPP and Notch endoproteolysis (Steiner, H. et al (1999) J Biol Chem 274:28669-73).

Pooled cell lines were generated and analyzed for PS endoproteolysis (PS proteins were detected by a combined immunoprecipitation/western blotting procedure (Steiner, H. et al., 1999; J Biol Chem 274:7615-8). Extracts from HEK 293 cells were prepared and subjected to immunoprecipitation using the polyclonal antibody 3027 to PS1 or 3711 to PS2 (Steiner, H. et al., 1999; J Biol Chem 274:7615-8). Following gel electrophoreses, immunoprecipitated PS proteins were identified by immunoblotting using the monoclonal antibody BI.3D7 [to PS1; (Steiner, H. et al., 1999; J biol Chem 274:7615-8)] or BI.HF5c [to PS2; (Steiner, H. et al., 1999; J Biol Chem 274:7615-8)]. As a control, untransfected cells expressing endogenous presenilins were investigated as well. Consistent with previous results (Thinakaran, G., et al (1996) Neuron 17:181-90), almost no PS1 holoprotein could be identified in cells expressing endogenous PS1 (FIG. 1B). In contrast, cell lines overexpressing ectopic PS 1 accumulated the PS 1 holoprotein (Figure1B). Efficient endoproteolysis of PS1 holoproteins was observed in cell lines expressing the G384A, G3841, G384P and G384W mutants FIG. 1B). In contrast, expression of G384K and G384D resulted in reduced amounts of the PS1 CTF indicating diminished PS 1 endoproteolysis by the introduction of charged amino acids at this position. As reported before (Wolfe, M. S. et al., 1999; Nature 398:513-7), the PS 1 D385A mutant also accumulated as an uncleaved holoprotein (FIG. 1B).

Stable expression of PS derivatives results in the displacement of endogenous presenilins (PS1 and PS2) (Thinakaran, G. et al., 1997; J Biol Chem 272:28415-22) and is a prerequisite for functional expression of exogenous presenilins. In order to prove if the G384 mutant PS1 variants displace endogenous presenilins, we next analyzed the expression of PS2 (PS proteins were detected by a combined immunoprecipitation/western blotting procedure (Steiner, H. et al., 1999; J Biol Chem 274:7615-8). Extracts from HEK 293 cells were prepared and subjected to immunoprecipitation using the polyclonal antibody 3027 to PS1 or 3711 to PS2 (Steiner, H. et al., 1999; J Biol Chem 274:7615-8). Following gel electrophoresis, immunoprecipitated PS proteins were identified by immunoblotting using the monoclonal antibody BI.3D7 [to PS1; (Steiner, H. et al., 1999; J Biol Chem 274:7615-8)] or BI.HF5c [to PS2; (Steiner, H. et al., 1999; J Biol Chem 274:7615-8). As demonstrated in FIG. 1B, overexpression of all PS1 G384 mutants as well as PS1 D385A and wt PS1 led to an almost complete displacement of endogenous PS2 CTFs.

We next investigated the influence of the PS1 G384 mutations on βAPP processing (To detect βAPP CTFs cell lysates were immunoblotted with antibody 6687 (raised to the last 20 C-terminal amino acids of βAPP). For the analysis of radiolabeled βAPP metabolites, HEK293 cells were metabolically labeled with 400 μCi³⁵S-methionine (Promix, Amersham) for 16 h in methionine-free MEM containing 5% dialyzed serum. Aβ species were immunoprecipitated from conditioned media with antibody 3926 [to Aβ 1-42 (Wild-Bode, C. et al., 1997; J Biol Chem 272:16085-8)] and separated on a previously described Tris-Bicine gel system (Wiltfang, J. et al; 1997; Electrophoresis 18:527-32), which allows the specific identification of Aβ40 and Aβ42 based on their characteristic running behavior. Quantitation of Aβ species was performed both by phosphoimager analysis and by a previously described ELISA (Steiner, H. et al, 1998; J Biol Chem 273:32322-31). Antibody 5313 (Steiner, H. et al., 1999; J Biol Chem 274:28669-73) was used to immunopreciptate the βAPP holoprotein from cell lysates or APPs from conditioned media). Since the aspartate mutations lead to an accumulation of βAPP CTFs probably due to reduced γ-secretase activity (DeStrooper, B. et al., 1998; Nature 391:387-90, Wolfe, M. S. et al., 1999; Nature 398:513-7), we first analyzed the levels of these amyloidogenic precursors. A significant accumulation of βAPP C-terminal fragments was observed in cell lines expressing the PS1 derivatives G3841I, G384P, G384W, G384K and G384D. These fragments accumulated as in cells expressing the PS 1 D385A mutant (FIG. 1C). In contrast, cells expressing the G384A mutation generate much lower levels of βAPP CTFs (FIG. 1C). This may indicate that all mutations except the FAD associated G384A variant are loss of function mutations, which directly interfere with γ-secretase activity.

Mutations of G384 Affect Aβ Production

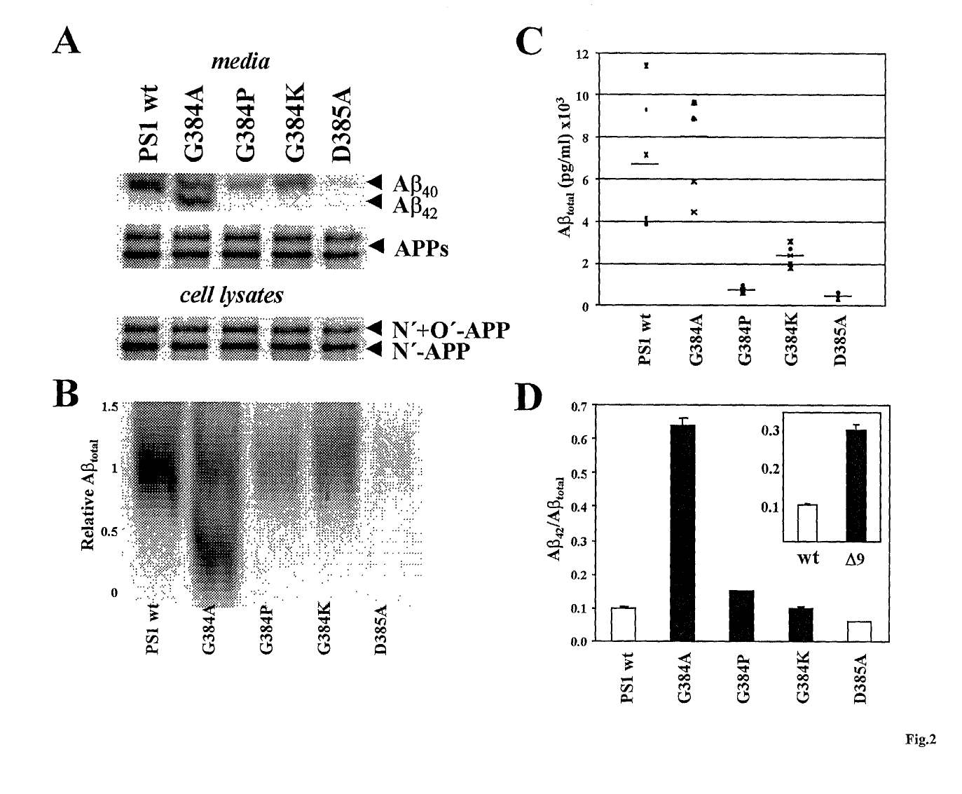

Since G384 mutations can affect βAPP CTF generation, we analyzed the functional consequences of these variants on Aβ generation. For further analysis, the mutations were divided into three groups according to their effects on PS endoproteolysis and βAPP CTF accumulation: (I) G384A: a natural occuring FAD associated mutation (Cruts, M. et al., 1995; Hum Mol Genet 4:2363-71, Tanahashi, H. et al., 1996; Neurosci Lett 218:139-41) which undergoes PS endoproteolysis and accumulates low levels of βAPP CTFs; (II) G384P, G3841I and G384W: artificial mutants which do not affect PS endoproteolysis and cause the accumulation of high levels of βAPP CTFs; (III) G384K and G384D: artificial mutants that undergo reduced PS endoproteolysis and accumulate high levels of βAPP CTFs. For further analysis one mutant from each group was chosen (G384A from group I, G384P from group II and G384K from group III). Cell lines were labeled with ³⁵S-methionine and conditioned media were investigated for Aβ levels. The FAD associated G384A mutant produced substantial amounts of total Aβ, including very high amounts of Aβ42 [Figure 2A; see (D) for quantitation]. In contrast, the proline and lysine substitution significantly reduced total Aβ production (FIG. 2A). In agreement with previous results (Wolfe, M. S. et al., 1999; Nature 398:513-7) the PS1 D385A mutation strongly inhibited total Aβ production (FIG. 2A). All mutations analyzed did not affect α-secretory processing or expression of the βAPP holoprotein (FIG. 2A).

Aβ levels were quantitated from conditioned media of ³⁵S-methionine labeled cell lines by phosphorimaging (FIG. 2B). Independent quantitations were obtained by analyzing Aβ concentrations in conditioned media of unlabeled cells using a previously described ELISA (FIG. 2C). Both assays confirmed that the proline and lysine mutation decreased Aβ production.

Finally, we quantitated the levels of Aβ42 and Aβ40 from conditioned media of ³⁵S-methionine labeled cell lines by phosphorimaging and calculated the Aβ42/Aβ total ratios. As shown in FIG. 2D, this revealed an extreme increase of the Aβ42/Aβ total ratio for the FAD associated G384A mutation, whereas the other mutations showed little or no effect. The approximate 6 fold increase of Aβ42 production is substantially higher then the Aβ42 production driven by the PS1 Δexon9 mutation, which is known to induce much higher Aβ42 levels than the usual point mutations (32; FIG. 2D, insert). Therefore the G384A mutation causes an exceptional increase of abnormal Aβ42 generation.

In contrast to the mutagenesis of the critical aspartate residue 385, mutagenesis of the neighboring glycine residue can therefore have two fundamentally different effects. Depending on the amino acid inserted, it can promote very high levels of pathological Aβ42 generation or reduce total Aβ production.

Interestingly, lack of PS 1 endoproteolysis does not necessarily result in the lack of γ-secretase activity as proposed previously (Wolfe, M. S. et al., 1999; Nature 398:513-7) since the G384P mutant significantly reduced Aβ generation (and Notch endoproteolysis; see below) although it undergoes endoproteolysis. In contrast, the G384K mutation blocked its endoproteolysis very similarly to the aspartate mutations and inhibited βAPP and Notch endoproteolysis (see below). This lack of correlation between PS endoproteolysis and βAPP/Notch cleavage is also supported by our previous findings that uncleaved PS holoproteins can support Aβ production (cDNAs encoding PS1 G384A, PS1 G2861I, PS1 G286P, PS1 G384W, PS1 G384K and PS1 G384D were constructed by oligonucleotide-directed mutagenesis using the polymerase chain reaction as described previously (Steiner, H. et al., 1999; Biochemistry 38:14600-5). The respective PCR products were cloned into EcoRI/Xhol restriction sites of the expression vector pcDNA3.1 containing a zeocin resistance gene (Invitrogen) and sequenced to verify successful mutagenesis (Song, W. et al., 1999; Proc Natl Acad Sci USA 96:6959-63).

Mutations at Amino Acid 384 Affect Notch Endoproteolysis

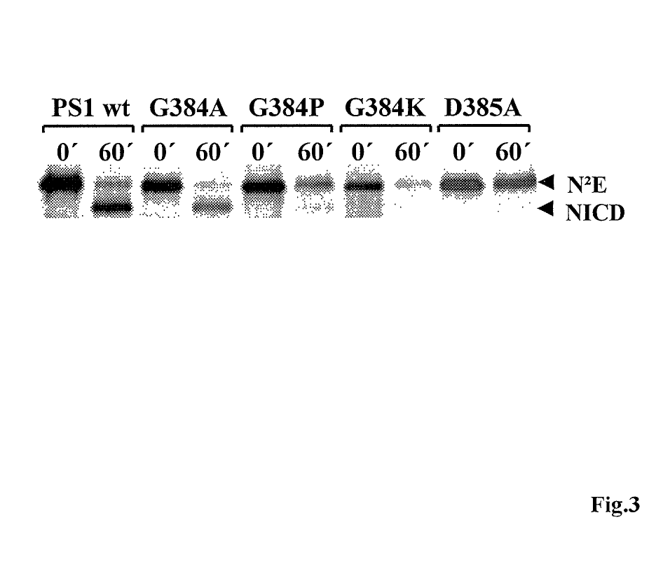

Since it has been shown previously that PS1 and PS2 are required to facilitate endoproteolysis of Notch (DeStrooper, B. et al., 1999; Nature 398:518-22, Steiner, H. et al., 1999; J Biol Chem 274:28669-73, Capell, A. et al., 2000; Nat Cell Biol 2:205-11, Ray, W. J. et al., 1999; J Biol Chem 274:36801-7, Song, W. et al., 1999; Proc Natl Acad Sci USA 96:6959-63), we now investigated whether expression of PS1 G384A, G384P and G384K affects NICD formation (HEK293 cells stably expressing Swedish mutant βAPP and PS1 derivatives were stably transfected with the previously described expression plasmid containing myc-tagged Notch E (NE) cDNA (Steiner, H. et al., 1999; J Biol Chem 274:28669-73)(Schroeter, E. H. et al., 1998; Nature 393:382-6). Transfected cells were cultured as described (HEK 293 cells stably expressing PS1 G384A, PS1 G2861I, PS1 G286P, PS1 G38W, PS1 G384K, PS1 G384D were generated by transfection of HEK 293 cells stably expressing βAPP containing the Swedish mutation (Citron, M., et al., 1992; Nature 360:672-4). Transfected HEK 293 cells were cultured in DMEM supplemented with 10% fetal bovine serum, 1% penicillin/streptomycine, 200μg ml⁻¹ G418 (to select for βAPP expression) and 200μg ml⁻¹ zeocin (to select for presenilin expression) except that 100 μg ml⁻¹ hygromycin was added to the culture medium to select for Notch expression. To analyze cleavage of N E, cells were starved for 1 h in methionine- and serum-free MEM, metabolically labeled with 300 μCi ³⁵S-methionine (Promix, Amersham) for 15 min, and chased for 1 h in medium containing excess amounts of unlabeled methionine. Cell extracts were prepared and Notch derivatives were immunoprecipitated using the antimyc antibody 9E10 as described (Steinter, H. et al., 1999; J Biol Chem 274:28669-73). HEK 293 cells expressing endogenous PS 1 or the indicated mutant derivatives were stably co-transfected with the NotchΔE cDNA construct described previously (Steiner, H. et al., 1999; J Biol Chem 274:28669-73, Schroeter, E. H. et al., 1998; Nature 393:382-6) and analyzed for Notch endoproteolysis. In control cells expressing wt PS 1, proteolytic release of NICD from NotchΔE was observed during the chase period (FIG. 3), which is consistent with previous results (Steiner, H. et al., 1999; J Biol Chem 274:28669-73, Capell, A. et al., 2000; Nat Cell Biol 2:205-11). Similarly, substantial NICD production was observed in cell lines expressing the FAD associated G384A mutant, which is also in line with previous results (Kulic, L. et al., 2000; Proc Natl Acad Sci USA 96:6959-63). In contrast, overexpression of PS1 G384P or PS1 G384K showed significantly reduced proteolytic generation of NICD (FIG. 3). Consistent with previous results (Song, W. et al., 1999; Proc Natl Acad Sci USA 96:6959-63), an almost complete block of NICD generation was observed in the cell line expressing PS1 D385A (FIG. 3). Moreover, the cDNA construct G384K did not rescue the sel-12 mutant phenotype upon transgenic expression in the worm (data not shown), independently confirming a loss of function in an in vivo system. Therefore, the mutagenesis of codon 384 to proline or lysine not only affects Aβ generation but also abolishes efficient NICD generation and consequently Notch signaling. However, the mutations at amino acid 384 behave completely differently from the previously described mutations of codon 286 (Kulic, L. et al., 2000; Proc Natl Acad Sci USA 96:6959-63). Insertion of charged amino acids at position 286 differentially affects Aβ production and NICD generation (Kulic, L. et al., 2000; Proc Natl Acad Sci USA 96:6959-63). In contrast, mutations at G384 either inhibit both, Aβ production and NICD generation, or specifically increase Aβ42 production without affecting Notch endoproteolysis. Therefore it is likely that G384 may directly affect the active center of presenilins (Wolfe, M. S. et al., 1999; Nature 398:513-7, Li, Y.-M., et al., 2000; Nature 405:689-94, Esler, W. P., et al., 2000; Nature Cell Biol 2:428-433).

G384/D385 are Part of a Motif Conserved in Bacterial Aspartyl Proteases

The above described data demonstrated that G384 is functionally important for γ-secretase mediated cleavage of βAPP and Notch. Moreover, Selkoe and Wolfe as well as Li et al. suggested that PS proteins are unusual aspartyl proteases (Li, Y. -M., et al., 2000; Nature 405:689-94, Esler, W. P., et al., 2000; Nature Cell Biol 2:428-433, Selkoe, D. J. and Wolfe, M. S., 2000; Proc Natl Acad Sci USA 97:5690-5692, Wolfe, M. S. et al., 1999; Biochemistry 38:11223-30), which are identical with the γ-secretase activity. However, so far no homology of presenilins to any known aspartyl protease was found (DeStrooper, 2000; J Cell Science 113:1857-70). Based on our data we hypothesized that the glycine residue together with the critical aspartate residue may be functionally conserved during evolution. In order to prove that, we first checked whether glycine 384 is present in all known presenilins. Indeed glycine 384 was found to be conserved in all members of the presenilin family, including the very distant worm homologue spe-4 (FIG. 4A). Furthermore even in the putative presenilin from Arabidopsis, the critical glycine is conserved (FIG. 4A). We therefore searched databases for a minimal conserved sequence motif in other proteases. For the search we used the sequence RLGFGDF. This sequence is derived from spe-4, the most distant member of presenilins. By using this sequence we thought to identify ancient aspartyl proteases, which may correspond to evolutionary ancestors of a novel family of bi-lobed aspartyl proteases. Strikingly, this search revealed numerous members of a novel family of bacterial aspartyl proteases (FIG. 4A). These proteases belong to the recently described family of type 4 prepilin peptidases (TFPP) (LaPointe, C. F. and Taylor R. K., 2000; J Biol Chem 275:1502-10). The TFPPs have 8 TM domains and two critical aspartate residues located close to TM3 and TM6 (FIG. 4B). Mutagenesis of the critical aspartates blocks the proteolytic function of the TFPPs (LaPointe, C. F. and Taylor R. K., 2000; J Biol Chem 275:1502-10). The conserved motif including the critical glycine residue is observed around the C-terminal but not the N-terminal aspartate residue (FIG. 4B), which again is consistent with the conservation of the same motif in TM7 but not TM6 of all members of the presenilin family. Moreover, TFPPs not only contain a very similar active site but also mediate an endoproteolytic cleavage, which is reminiscent to the cleavage reactions supported/mediated by PSs. TFPPs are known to remove leader peptides of selected substrates by cleaving between hydrophobic and hydrophilic domains close to the cytoplasmic site of the membrane (LaPointe, C. F. and Taylor R. K., 2000; J Biol Chem 275:1502-10), a cleavage reaction, which is strikingly similar to the γ-secretase cleavage of Notch and βAPP (Haass, C. and DeStrooper, B., 1999; Science 286:916-9). Moreover, it has been demonstrated that the C-terminal aspartate residue (D189) in TcpJ, a typical member of the TFPPs, is less tolerant to mutagenesis as the N-terminal aspartate residue of TcpJ (D125; 48). This is again similar to PS1, since mutagenesis of D257 does not necessarily affect PS function on PAPP processing as dramatically as the corresponding mutations at D385 (Song, W. et al., 1999; Proc Natl Acad Sci USA 96:6959-63). Based on these data we suggest that an aspartyl protease activity of presenilins could have evolved from ancient bacterial proteases. TFPPs and PSs appear to share the common protease consensus site motif (FIG. 4B) G(A)X′GDX″ (X′=variable; X″=F>I>V>L; bold: amino acids conserved in all members of both families). The only exception is a putative prepilin like processing peptidase (accession CAB73090), which contains an alanine immediately N-terminal to the aspartate. However, this agrees with our data since an alanine at the corresponding position of PS1 (G384A) does not inhibit endoproteolysis βAPP and Notch. In fact, PS1 G384A even severely enhances Aβ42 production (see FIGS. 2A and D). During evolution a new family of aspartyl proteases emerged, which now occurs from procaryotes to metazoans. In higher organisms these polytopic “PS-proteases” are required to cleave βAPP, Notch, and Ire1 either within the membrane or close to the cytoplasmic site of the membrane. This protease activity occurs as a high molecular weight complex (Capell, A. et al., 1998; J Biol Chem 273:3205-11; Yu, G. et al., 1998; J Biol Chem 273:16470-5; Thinakaran, G. et al, 1998; Neurobiol Dis 4:438-53; Li, Y. -M. et al., 2000; Proc Natl Acad Sci USA 97:6138-6143), which may contain additional proteins required for its function.

The present invention is not to be limited in scope by the exemplified embodiments which are intended as illustrations of single aspects of the invention. Indeed various modifications of the invention in addition to those shown and described herein will become apparent to those skilled in the art from the foregoing description and accompanying drawings. Such modifications are intended to fall within the scope of the appended claims.

What is claimed is:

1. A protein or peptide comprising the consensus motif X¹-Leu-Gly-X²-Gly-Asp-Phe-X³ or X⁴-X⁵-X⁶-X⁷-Gly-Asp-X⁸-X⁹, wherein X¹, is Lys or Arg; X² is Lys, Phe, Met or Leu; X³ is Ile, Tyr or Val; X⁴ is Val, Gly, Ala; X⁵ is Met, Phe, Val, Ile, Leu; X⁶ is Gly or Ala, X⁷ is Tyr, Gly, Asp, Phe or His; X⁸ is Ile, Leu, Pro, Val or Phe; and X⁹ is Lys, Ala or Ile.

2. The protein or peptide according to claim 1 comprising the consensus motif X¹-Leu-Gly-X²-Gly-Asp-Phe-X³ wherein X¹, is Lys or Arg; X² is Lys, Phe, Met or Leu and X³ is Ile, Tyr or Val.

3. The protein or peptide according to claim 1 comprising the consensus motif X⁴-X⁵-X⁶-X⁷-Gly-Asp-X⁸-X⁹, wherein X⁴ is Val, Gly, Ala; X⁵ is Met, Phe, Val, Ile, Leu; X⁶ is Gly or Ala, X⁷ is Tyr, Gly, Asp, Phe or His; X⁸ is Ile, Leu, Pro, Val or Phe and X⁹ is Lys, Ala or Ile.

4. The peptide according to any one of claims 1 or 2, which consists of the consensus motif X¹-Leu-Gly-X²-Gly-Asp-Phe-X³, wherein X¹, is Lys or Arg; X² is Lys, Phe, Met or Le and X³is Ile, Tyr or Val.

5. The peptide according to any one of claims 1 or 3, which consists of the consensus motif X⁴-X⁵-X⁶-X⁷-Gly-Asp-X⁸-X⁹, wherein X⁴ is Val, Gly, Ala; X⁵ is Met, Phe, Val, Ile, Leu; X⁶ is Gly or Ala, X⁷ is Tyr, Gly, Asp, Phe or His; X⁸ is Ile, Leu, Pro, Val or Phe and X⁹ is Lys, Ala or Ile.

6. The peptide according to claim 2, which consists of the consensus motif Lys-Leu-Gly-Leu-Gly-Asp-Phe-Ile.

7. The peptide according to claim 3, which consists of the the consensus motif Gly-Met-Gly-Tyr-Gly-Asp-Phe-Lys.

8. The protein according to claim 1, wherein the protein is an aspartyl-protease.

9. The aspartyl-protease according to claim 8, which comprises the consensus motif Lys-Leu-Gly-Leu-Gly-Asp-Phe-Ile.

10. The aspartyl-protease according to claim 8, which comprises the consensus motif Gly-Met-Gly-Tyr-Gly-Asp-Phe-Lys.

11. An aspartyl-protease comprising the consensus motif Gly-X¹-X²-Gly-Asp-X³; wherein X¹ is Ala or no amino acid; X² is any amino acid and X³ is Phe, Ile, Val or Leu.

12. The peptide consisting of the consensus motif Gly-X¹-X²-Gly-Asp-X³; wherein X¹ is Ala or no amino acid; X² is any amino acid and X³ is Phe, Ile, Val or Leu.

13. A nucleic acid encoding any one of the proteins or peptides according to any one of claims 1 to 3 or 6 to 12.

14. A method of screening for substances capable of inhibiting an aspartyl-protease comprising:
a) culturing cells expressing (i) the aspartyl-protease according to any one of claims 8 to 11 and (ii) a membrane-associated fusion protein comprising a substrate with the specific cleavage site of said aspartyl-protease and a reporter;
b) incubating said cells with a test substance;
c) measuring the amount of cleaved-off reporter; and
d) comparing the value obtained to the value obtained in the absence of the test compound.

15. The method according to claim 14, wherein the cells in step (a) express a fusion protein comprising the cleavage site of a γ-secretase.

16. The method according to claim 14, wherein the cells in step (a) express a fusion protein comprising the cleavage site of a presenilinase.

17. The method according to claim 14, wherein the substrate of the fusion protein comprises amyloid β or a fragment thereof.

18. The method according to claim 14, wherein the substrate of the fusion protein comprises a fragment of amyloid precursor protein or a fragment thereof.

19. The method according to claim 14, wherein the substrate of the fusion protein comprises presenilin 1 or a fragment thereof.

20. The method according to claim 14, wherein the substrate of the fusion protein comprises presenilin 2 or a fragment thereof.

21. The method according to claim 14, wherein the method is a high throughput screening method.

22. A method of screening for substances capable of identifying an inhibitor of a presenilinase or the autoproteolytic cleavage of presenilin comprising:
(a) culturing cells expressing (i) the protein or pepetide according to claim 1 and (ii) a membrane-associated fusion protein comprising a substrate with the specific cleavage site of said presenilinase and a reporter;
(b) incubating said cells with a test substance;
(c) measuring the amount of cleaved-off reporter; and
(d) comparing the value obtained to the value obtained in the absence of the test compound.

23. A method of screening for substances capable of inhibiting a γ-secretase comprising:
(a) culturing cells expressing (i) the protein or pepetide according to claim 1 and (ii) a membrane-associated fusion protein comprising a substrate with the specific cleavage site of said γ-secretase and a reporter;
(b) incubating said cells with a test substance;
(c) measuring the amount of cleaved-off reporter; and
(d) comparing the value obtained to the value obtained in the absence of the test compound.

24. A substance identifiable with a method according to claim 14 wherein said substance is capable of specifically inhibiting the proteolytic cleavage of a γ-secretase-substrate.

25. A substance identifiable with a method according to claim 14 wherein said substance is capable of specifically inhibiting the proteolytic cleavage of presenilin.

26. A pharmaceutical composition comprising a substance according to claim 24; and a pharmaceutically acceptable carrier or excipient.

27. A pharmaceutical composition comprising a substance according to claim 25; and a pharmaceutically acceptable carrier or excipient.

28. A pharmaceutical composition comprising a protein or peptide according to claim 1; and a pharmaceutically acceptable carrier or excipient.
